(19) United States
(12) Patent Application Publication
(10) Pub. No.: US 2006/0121112 A1
(43) Pub. Date: Jun. 8, 2006

(54) Topiramate pharmaceutical composition

(75) Inventors: Scott A. Jenkins, Downingtown, PA (US); Gary Liversidge, Westchester, PA (US)

Correspondence Address: SYNNESTVEDT & LECHNER, LLP, 2600 ARAMARK TOWER, 1101 MARKET STREET, PHILADELPHIA, PA, 191072950, US

(73) Assignee: Elan Corporation, plc, Lincoln Place (IE)

(21) Appl. No.: 11/297,737

(22) Filed: Dec. 8, 2005

Related U.S. Application Data
(60) Provisional application No. 60/634,301, filed on Dec. 8, 2004.

(51) Int. Cl. A61K 31/7008; A61K 9/22

(52) U.S. Cl. 424/468; 514/23

(57) ABSTRACT

A once daily controlled-release pharmaceutical formulation which contains therapeutic amounts of topiramate and which is capable of being administered to specific regions along the gastrointestinal tract used to treat various types of conditions, for example, partial seizures with or without secondarily generalized seizures, primary generalized tonic-clonic seizures, seizures associated with Lennox Gastaut Syndrome, migraines, and obesity.

### FIELD OF THE INVENTION

The present invention relates to a controlled-release topiramate pharmaceutical composition. More particularly, the present invention relates to a once-daily controlled-release pharmaceutical formulation which contains therapeutic amounts of topiramate and which is capable of being administered to specific regions along the gastrointestinal tract.

Topiramate is a sulfamate-substituted monosaccharide indicated as adjunctive therapy for partial seizures, with or without secondarily generalized seizures, and for primary generalized tonic-clonic seizures. Topiramate is also indicated as adjunctive therapy for seizures associated with Lennox Gastaut Syndrome. Topiramate is sold in the United States under the trade name TOPAMAX™ (Ortho-McNeil Pharmaceutical, Inc., Raritan, N.J., U.S.A.).

Topiramate is a relatively potent anticonvulsant and is structurally different from other antiepileptic drugs. It is derived from D-fructose and was developed initially as an antidiabetic drug. In animal models, it was found to have potent antiepileptic effects. Topiramate has multiple mechanisms of action. It exerts an inhibitory effect on sodium conductance, decreasing the duration of spontaneous bursts and the frequency of generated action potentials, enhances GABA by unknown mechanisms, inhibits the AMPA subtype glutamate receptor, and is a weak inhibitor of carbonic anhydrase.

In addition to its use in the treatment of seizures as described above, there is evidence that topiramate may also be effective as an anti-obesity agent, a blood pressure lowering agent, and a mood stabilizer, including use as an antimanic and antidepressant, and for the treatment of post-traumatic stress disorder, migraines, cluster headaches, and neuropathic pain. See, e.g., U.S. Pat. Nos. 6,191,117; 6,201,010; 5,753,693; 5,998,380; 6,319,903; 5,935,933; and 5,760,007; and patent Application Publication 2004/0002462 A1, each of which incorporated herein by reference in their entirety.

Topiramate pharmacokinetics is linear, producing a dose proportional increase in blood plasma concentration levels. Topiramate is absorbed rapidly after oral administration and has a bioavailability close to 100%. When administered at regular doses, food can delay but does not affect the extent of absorption. The time to peak blood levels is about 2 hours following a 400 mg oral dose. The volume of distribution ranges from about 0.6 to about 1.0 L/kg. Plasma protein binding is approximately 15%. Only 15% of topiramate is metabolized in the liver by the P-450 microsomal enzyme system. None of the metabolites has antiepileptic action, and the majority of the drug (i.e., about 85%) is excreted unchanged in the urine. However, metabolism is much more extensive in patients on polytherapy, presumably as a result of enzyme induction. In patients with renal failure, doses may need to be reduced. The elimination half-life ranges from about 18 to about 23 hours and is independent of dose over the normal clinical range. In experimental settings, no tolerance to topiramate has been recorded. Topamax™ is traditionally dosed at about 400 mg/day in two divided dosage units.

Knowledge of the relative rate and extent of absorption of a drug from different regions of the gastrointestinal tract may be crucial in the timely, cost-effective, and rational development of a controlled-release formulation. The properties of a drug cannot be used to predict precisely the optimal site of absorption and often drugs are dropped from development pipelines of pharmaceutical companies because of a lack of such information. The traditional approach is to perform in vitro dissolution tests to provide an understanding of how the formulation might be handled by the body. This is followed by a pharmacokinetic study in human volunteers, which often results in equivocal findings, thus making optimization of the formulation difficult.

A more rational approach is to obtain detailed information about how well a drug is absorbed from the key residence areas of the gastrointestinal tract in order to optimize the delivery of the drug at lower doses while achieving bioavailability associated with a greater dose.

### SUMMARY OF THE INVENTION

One aspect of the present invention is the provision of a controlled-release pharmaceutical composition comprising topiramate which is capable of delivering therapeutic amounts of topiramate to the serum of an animal, preferably a human, through the animal's gastrointestinal tract. More specifically, the present invention relates to a controlled-release pharmaceutical composition comprising topiramate which is capable of delivering therapeutic amounts of topiramate to the proximal small bowel, distal small bowel and/or colonic regions of the gastrointestinal tract. The properties of the composition may be achieved, for example, by the use of a controlled-release pharmaceutical composition comprising topiramate which comprises the following components, each of which includes topiramate: (A) an immediate-release (IR) component comprising from about 5 mg to about 250 mg of topiramate which is released within about 1 hour after administration; and (B) a delayed-release (DR) component which releases from about 5 mg to about 250 mg of topiramate in the body over a period of time of about 6 hours to about 24 hours after administration.

Another aspect of the present invention is the provision of a controlled-release pharmaceutical composition comprising topiramate which is capable of delivering therapeutic amounts of topiramate in an oral dosage form.

Another aspect of the present invention is the provision of a controlled-release pharmaceutical composition comprising topiramate which is capable of delivering therapeutic amounts of topiramate in a once-daily formulation. The controlled-release properties of the composition may be achieved, for example, by the use of pharmaceutically acceptable erodable formulations, diffusion controlled formulations or osmotically controlled formulations. Formulations may include, but are not limited to, capsules, caplets, tablets, matrices, microcapsules or microgranules, and the like.

Another aspect of the present invention is the provision of a controlled-release pharmaceutical composition comprising topiramate in combination with one or more additional active agents.

Applicants have surprisingly discovered from the studies described more fully below that the administration of topiramate at various locations along the gastrointestinal tract demonstrates differential bioavailability. Specifically, topiramate exhibits a higher bioavailability in colonic regions of the gastrointestinal tract as compared with the stomach. As a result of this differential bioavailability, applicants have found that controlled-release forms of topiramate can be specifically designed to take advantage of this differential bioavailability. For example, a dosage can be designed wherein the delayed-release component contains an amount of topiramate less than that of the immediate-release component but that nonetheless achieves a blood plasma concentration equivalent to that of the immediate-release component. Alternatively, a dosage can be designed so that the delayed-release component contains an amount of topiramate equal to that of the immediate-release component yet would achieve a blood plasma concentration greater than that of the immediate-release component. The discovery of the differential bioavailability of topiramate allows a more rational drug design based upon the specific therapeutic profiles to be achieved.

The controlled-release properties of the composition may be achieved, for example, by the use of a pharmaceutical compositions and dosage forms having a substantially continuous or pulsatile-release profile comprising topiramate. Pharmaceutical compositions and dosage forms having pulsatile-release profiles may comprise the following components: (A) an immediate-release component comprising from about 5 mg to about 250 mg of topiramate which is released within about 1 hour after ingestion; and (B) a delayed-release component comprising from about 5 mg to about 250 mg of topiramate which is released in the body at about 6 hours, about 9 hours, about 12 hours or about 18 hours after the release of the immediate release component. Pulsatile-release compositions may optionally include additional delayed-release components comprising from about 5 mg to about 250 mg of topiramate which is released in the body subsequent to the release of the first delayed-release component. When a relatively constant blood plasma concentration profile is desired, lower doses of topiramate may be provided in the delayed-release component(s) relative to the immediate release component due to the higher bioavailability of topiramate in colonic regions of the gastrointestinal tract.

Another aspect of the present invention is the provision of a pulsatile-release pharmaceutical composition comprising topiramate which is capable of delivering therapeutic amounts of topiramate to the proximal small bowel, distal small bowel and colonic regions of the gastrointestinal tract in a once-daily solid oral-dosage form. The amounts of topiramate that are delivered to each region of the gastrointestinal tract are selected in accordance with the differential bioavailability profile of topiramate so that the desired blod plasma concentration profile is achieved.

A further aspect of the present invention is a method for treating partial seizures, with or without secondarily generalized seizures, primary generalized tonic-clonic seizures, seizures associated with Lennox Gastaut Syndrome, or migraines by administering a controlled-release pharmaceutical composition comprising topiramate which is capable of delivering therapeutic amounts of topiramate in an oral dosage formulation to the proximal small bowel, distal small bowel and colonic regions of the gastrointestinal tract. Preferably, the oral dosage formulation has a controlled-release feature that permits the release of therapeutic amounts of topiramate over a 24-hour period of time.

Another aspect of the present invention is a method of treating obesity and obesity related disorders and syndromes, including Syndrome X, by administering a pharmaceutical composition comprising a controlled-release form of topiramate and a sympathomimetic agent which is capable of delivering therapeutic amounts of topiramate in an oral dosage formulation to the proximal small bowel, distal small bowel and colonic regions of the gastrointestinal tract. In one embodiment, the oral dosage formulation has a controlled-release feature that permits the release of therapeutic amounts of topiramate over a 24-hour period of time.

### DETAILED DESCRIPTION OF THE INVENTION

The composition of the present invention comprises a controlled-release pharmaceutical composition comprising topiramate which is capable of delivering therapeutic amounts of topiramate to the proximal small bowel, distal small bowel or colonic regions of the gastrointestinal tract of an animal.

One embodiment of the present invention provides a controlled-release pharmaceutical composition comprising topiramate which comprises the following components, each of which includes topiramate: (A) an immediate-release (IR) component comprising from about 5 mg to about 250 mg of topiramate which is released within about 1 hour after administration; and (B) a delayed-release (DR) component comprising from about 5 mg to about 250 mg of topiramate which is released in the body over a period of time of about 6 hours to about 24 hours after administration.

Another embodiment of the present invention provides a pulsatile-release pharmaceutical composition comprising topiramate which comprises the following components: (A) an immediate-release component comprising from about 5 mg to about 250 mg of topiramate which is released within about 1 hour after ingestion; and (B) a delayed-release component comprising from about 5 mg to about 250 mg of topiramate which is released in the body at about 6 hours, about 9 hours, about 12 hours or about 18 hours after the release of the immediate-release component; and optionally (C) one or more subsequent delayed-release components comprising from about 5mg to about 250mg of topiramate which is released in the body at about 6 hours, about 9 hours, about 12 hours or about 18 hours after the release of the first delayed-release component. Reduced amounts of topiramate may be provided in the delayed-release components as compared with the immediate-release component without a decrease in blood plasma concentration due to greater bioavailability of topiramate in colonic regions of the gastrointestinal tract.

In one embodiment, there is provided a multiparticulate modified-release composition having a first component comprising a first population of topiramate-containing particles and a second component comprising a second population of topiramate-containing particles. The first component may be an immediate-release component or a delayed-release component. The active ingredient-containing particles of the second component may be coated with a modified-release coating or provided in a controlled-release matrix material. In embodiments in which the topiramate-containing particles of the second component are coated with a modified-release coating, the coating applied to the second population of particles causes a lag time between the release of topiramate from the first population of particles and the release of topiramate from the second population of particles. Similarly, the presence of a modified-release matrix material in the second population of particles causes a lag time between the release of topiramate from the first population of particles and the release of topiramate from the second population of particles. The duration of the lag time may be varied by altering the composition and/or the amount of the modified-release coating and/or altering the composition and/or amount of modified-release matrix material utilized. Thus, the duration of the lag time can be designed to achieve a desired plasma profile. Following oral delivery, the composition in operation is capable of delivering the active ingredient or active ingredients in a pulsatile manner.

The multiparticulate modified-release composition of the invention may comprise more than two components. In such embodiments, the release of topiramate from the second and subsequent components is modified such that there is a lag time between the release of topiramate from the first component and each subsequent component. The number of pulses in the profile arising from such a composition in operation will depend on the number of topiramate-containing components in the composition. For example, a composition containing three active topiramate-containing components will give rise to three pulses in the profile.

The multiparticulate modified-release composition of the invention may further comprise one or more additional active ingredients that are compatible with topiramate and, if more than one additional active ingredient, each other. In one embodiment, the multiparticulate modified-release composition of the invention comprises a therapeutically effective amount of the controlled-release form of topiramate of the present invention in combination with a sympathomimetic agent. Sympathomimetic agents are a class of drugs known for their ability to mimic or alter stimulation of the sympathetic nervous system (e.g., stimulates the peripheral nervous system) of an organism (e.g., mimic the stimulation naturally effected by physical activity, psychological stress, generalized allergic reaction and other situations in which the organism is provoked). Sympathomimetic agents include phenylethylamine, epinephrine, norepinephrine, epinine, dopamine, dabutamine, nordefrin, ethylnorepinephrine, isoproterenol, protokylol, isoetharine, metaproterenol, terbutaline, metaraminol, phenylephrine, tyramine, hydroxyamphetamine, methoxyphenamine, methoxamine, albuterol, amphetamine, methamphetamine, benzphetamine, ephedrine, phenylpropanolamine, mephentermine, phentermine, chlorphentermine, fenfluramine, tuaminoheptane, propylhexedrine, diethylpropran, phenmetrazine, and phendimetrazine.

In one embodiment, the sympathomimetic agent has anorexient properties (e.g., suppresses appetite) or is anorectic without significant toxicity to a subject or patient at therapeutically effective doses. In another embodiment, the sympathomimetic agent has anorexient properties or is anorectic without loss of efficacy or without adverse or undesirable side effects to a subject or patient at therapeutically effective doses when prescribed in combination with topiramate.

In yet another embodiment, the sympathomimetic agent is phentermine or a phentermine-like compound. Phentermine hydrochloride, also known as 1,1 -Dimethyl-2-phenethylamine hydrochloride, is a white, odorless, hygroscopic, crystalline powder which is soluble in water and lower alcohols, slightly soluble in chloroform and insoluble in ether. It is prescribed as an anorectic agent and is readily available in capsule or tablet form. A phentermine-like compound is a compound structurally related to phentermine (e.g., an analog or derivative) which maintains an anorectic activity similar to that of phentermine. A preferred phentermine-like compound is chlorphentermine.

Combination therapy according to the present invention provides an effective and efficient treatment for bringing about weight loss while simultaneously enabling a reduction in the effective dosage of each drug administered and minimizing the potential side effects of each individual drug. Administering topiramate with phentermine according to the invention, for example, is very effective in bringing about weight loss using a daily dose of topiramate that is at least half of the recommended daily dose of the drug (400 mg) and a daily dose of phentermine that is also significantly reduced relative to the recommended daily dose (from 37.5 mg daily to 5-15 mg daily). At the same time, weight loss is achieved efficiently and the common side effects of each drug are substantially reduced.

In yet another embodiment, the sympathomimetic agent is amphetamine or an amphetamine-like compound. As used herein, an “amphetamine-like compound” is a compound structurally related to amphetamine (e.g., an analog or derivative) which maintains an anorectic effect of amphetamine.

In yet another embodiment, the sympathomimetic agent is phenmetrazine or a phenmetrazine-like compound. As defined herein, a “phenmetrazine-like compound” is a compound structurally related to phenmetrazine (e.g., an analog or derivative) which maintains an anorectic effect of phenmetrazine. A preferred phenmetrazine-like compound is phendimetrazine. Analogs and/or derivatives of the compounds of the present invention can be tested for their ability to suppress appetite in a subject.

In an exemplary embodiment, the sympathomimetic agent is selected from the group consisting of amphetamine, methamphetamine, phentermine, benzphetamine, phenylpropanolamine, chlorphentermine, diethylpropion, phenmetrazine, and phendimetrazine. In another exemplary embodiment, the sympathomimetic agent is phentermine. It is also within the scope of the present invention to utilize other sympathomimetic agents including caffeine, pseudoephedrine, methylphenidate and other CNS stimulants.

The sympathomimetic drug is prescribed at a dosage that is at most that which is routinely used by the skilled artisan (e.g., physician) to promote the desired therapeutic effect of the drug, when the drug is used as a monotherapy. The sympathomimetic drug may be prescribed, for example, at a dose of 5-1000, 10-1000, 20-1000, and 25-50 mg daily. In one embodiment, a maintenance dose that is co-administered with topiramate is from about 50 mg to about 400 mg daily. In another embodiment, the maintenance dose is from about 50 mg to about 250 mg daily. In yet another embodiment, the maintenance dose is from about 100 mg to about 250 mg daily. In a further embodiment, the maintenance dose is from about 100 mg to about 200 mg daily. By “maintenance dose” is meant an ongoing daily dose administered to a patient. This maintenance dose may be established at the outset or after gradually increasing the daily dose from an initial, low dosage, over an extended time period, e.g., on the order of several weeks.

The dosage should be individualized to obtain an adequate response with the lowest effective dose. While a common adult dose is 37.5 mg phentermine, administered daily either before breakfast or 1-2 hours after breakfast the dose may be adjusted to the patient's specific needs. For some patients 18.75 mg daily may be adequate, while in some cases it may be desirable to administer 18.75 mg twice a day.

In another aspect of the present invention, there is provided a method for effecting weight loss which involves treating a subject with a controlled-release form of topiramate in combination with a sympathomimetic agent such that the subject experiences weight loss. In one embodiment, the sympathomimetic agent is a compound having anorectic activity, for example, amphetamine, methamphetamine, benzphetamine, phentermine, chlorphentermine, diethylpropran, phenmetrazine, and phendimetrazine. In another embodiment, the sympathomimetic agent is phentermine.

The methods of the present invention also are effective against symptoms associated with Syndrome X. Accordingly, in another aspect the invention features methods for treating Syndrome X with a combination of a controlled-release form of topiramate and a sympathomimetic agent and such that at least one symptom associated with Syndrome X is beneficially affected. Moreover, the combination methods of the present invention have been shown to have additional beneficial effects, such as ameliorating sleep apnea and lowering blood pressure, blood glucose, blood lipid, and Hgb A1C levels. Accordingly, in another aspect of the invention are methods for treating at least one additional effect associated with obesity. In one embodiment, at least one side effect of obesity is treated with a combination of a controlled-release form of topiramate and a sympathomimetic agent such as, for example, phentermine.

A drug compound present in one component of the composition may also be accompanied by, for example, an enhancer compound or a sensitizer compound in the same or another component of the composition in order to modify the bioavailability or therapeutic effect of the drug compound.

As used herein, the term “enhancer” refers to a compound which is capable of enhancing the absorption and/or bioavailability of an active ingredient by promoting net transport across the GI tract in an animal, such as, for example, a human. Enhancers include, but are not limited to: medium chain fatty acids and salts, esters, and ethers and derivatives thereof, including, for example, glycerides and triglycerides; non-ionic surfactants, for example, those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more enhancers.

The proportion of topiramate contained in each component may be the same or different depending on the desired dosing regime. The topiramate may be present in the first component individually or in combination with the topiramate in the second component in any amount sufficient to elicit a therapeutic response. The topiramate (and optional other active ingredients) may be present either in the form of one substantially optically pure enantiomer or as a mixture, racemic or otherwise, of enantiomers.

Because the plasma profile produced by the multiparticulate modified-release composition upon administration is substantially similar to the plasma profile produced by the administration of two or more immediate-release dosage forms given sequentially, the multiparticulate controlled release composition of the present invention is particularly useful for administering active ingredients for which such plasma profiles are desired.

The present invention also provides solid oral dosage forms of topiramate comprising a composition according to the invention. The solid oral dosage forms of the present invention may further comprise a sympathomimetic agent such as, for example, phentermine.

The present invention further provides a method of treating an animal, particularly a human, in need of treatment comprising administering a therapeutically effective amount of a composition or solid oral dosage form according to the invention to provide pulsed or bimodal administration of topiramate.

The time release characteristics for the release of the topiramate from each of the components may be varied by modifying the composition of each component, including modifying any of the excipients or coatings which may be present. In particular, the release of the active may be controlled by changing the composition and/or the amount of the modified-release coating on the particles, if such a coating is present. If more than one modified-release component is present, the modified-release coating for each of these components may be the same or different. Similarly, when modified-release is facilitated by the inclusion of a modified-release matrix material, release of the topiramate may be controlled by the choice and amount of modified-release matrix material utilized. The modified-release coating may be present, in each component, in any amount that is sufficient to yield the desired delay time for each particular component. The modified-release coating may be preset, in each component, in any amount that is sufficient to yield the desired time lag between components.

The lag time or delay time for the release of the active ingredient from each component may also be varied by modifying the composition of each of the components, including modifying any excipients and coatings which may be present. For example, the first component may be an immediate-release component from which the topiramate is released substantially immediately upon administration. Alternatively, the first component may be, for example, a time-delayed immediate-release component in which the topiramate is released substantially immediately after a time delay. The second component may be, for example, a time-delayed immediate-release component as just described or, alternatively, a time-delayed sustained-release or extended-release component in which the topiramate is released in a controlled fashion over an extended period of time.

As will be appreciated by those skilled in the art, the exact nature of the plasma concentration curve will be influenced by the combination of all of the aforementioned factors. In particular, the lag time between the delivery (and thus also the onset of action) of the topiramate in each component may be controlled by varying the composition and coating, if present, of each of the components. Thus, by variation of the composition of each component and by variation of the lag time, numerous release and plasma profiles may be obtained. Depending on the duration of the lag time between the release of topiramate from each component and the nature of the release from each component (i.e. immediate release, sustained release etc.), the pulses in the plasma profile may be well separated and clearly defined peaks (e.g. when the lag time is long) or the pulses may be superimposed to a degree (e.g. in when the lag time is short).

In another embodiment, the multiparticulate modified-release composition according to the present invention has an immediate-release component and at least one modified-release component, the immediate-release component comprising a first population of topiramate-containing particles and the modified-release components comprising second and subsequent populations of topiramate-containing particles. The second and subsequent modified-release components may comprise a controlled-release coating. Additionally or alternatively, the second and subsequent modified-release components may comprise a modified-release matrix material. In operation, administration of such a multiparticulate modified-release composition having, for example, a single modified-release component results in characteristic pulsatile plasma concentration levels of the topiramate in which the immediate-release component of the composition gives rise to a first peak in the plasma profile and the modified-release component gives rise to a second peak in the plasma profile. Embodiments of the invention comprising more than one modified-release component give rise to further peaks in the plasma profile.

Any coating material which modifies the release of the active ingredient in the desired manner may be used. In particular, coating materials suitable for use in the practice of the invention include, but are not limited to: polymer materials, such as, for example, cellulose acetate phthalate, cellulose acetate trimaletate, hydroxypropylmethylcellulose phthalate, polyvinyl acetate phthalate, ammonio methacrylate copolymers such as those sold under the Trade Mark Eudragit® RS and RL, polyacrylic acid and polyacrylate and methacrylate copolymers, for example, those sold under the trademark Eudragit® S and L, polyvinyl acetaldiethylamino acetate, hydroxypropylmethylcellulose acetate succinate, and shellac; hydrogels and gel-forming materials, such as, for example, carboxyvinyl polymers, sodium alginate, sodium carmellose, calcium carmellose, sodium carboxymethyl starch, poly (vinyl alcohol), hydroxyethylcellulose, methyl cellulose, gelatin, starch, and cellulose based cross-linked polymers in which the degree of crosslinking is low so as to facilitate adsorption of water and expansion of the polymer matrix, hydroxypropylcellulose, hydroxypropyl-methylcellulose, polyvinylpyrrolidone, crosslinked starch, microcrystalline cellulose, chitin, aminoacrylmethacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (m. wt. about 5 k-5,000 k), polyvinylpyrrolidone (m. wt. about 10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. about 30 k-300 k), polysaccharides such as agar, acacia, karaya, tragacanth, algins and guar, polyacrylamides, Polyox® polyethylene oxides (m. wt. about 100 k -5,000 k), AquaKeep® acrylate polymers, diesters of polyglucan, crosslinked polyvinyl alcohol and poly N-vinyl-2-pyrrolidone, sodium starch glycolate (e.g. Explotab®; Edward Mandell C. Ltd.); hydrophilic polymers such as polysaccharides, methyl cellulose, sodium or calcium carboxymethyl cellulose, hydroxypropylmethylcellulose, hydroxypropylcellulose, hydroxyethylcellulose, nitro cellulose, carboxymethyl cellulose, cellulose ethers, polyethylene oxides (e.g. Polyox®, Union Carbide), methyl ethyl cellulose, ethylhydroxyethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof.

As will be appreciated by the person skilled in the art, excipients such as, for example, plasticisers, lubricants, solvents and the like may be added to the coating. Suitable plasticisers include, for example, acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

When the modified-release component comprises a modified-release matrix material, any suitable modified-release matrix material or suitable combination of modified-release matrix materials may be used. Such materials are known to those skilled in the art. The term “modified-release matrix material” as used herein includes, for example, hydrophilic polymers, hydrophobic polymers and mixtures thereof which are capable of modifying the release of an active ingredient dispersed therein in vitro or in vivo. Modified-release matrix materials suitable for the practice of the present invention include, but are not limited to, microcrystalline cellulose, sodium carboxymethylcellulose, hydroxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate and mixture thereof.

A multiparticulate modified-release composition according to the present invention may be incorporated into any suitable dosage form which facilitates release of the active ingredient in a pulsatile manner. Typically, the dosage form may be a blend of the different populations of active ingredient containing particles which make up the immediate-release and the modified-release components, the blend being filled into suitable capsules, such as hard or soft gelatin capsules. Alternatively, the different individual populations of active ingredient containing particles may be compressed (optionally with additional excipients) into mini-tablets which may be subsequently filled into capsules in the appropriate proportions. Another suitable dosage form is that of a multilayer tablet. In this instance the first component of the multiparticulate modified-release composition may be compressed into one layer, with the second component being subsequently added as a second layer of the multilayer tablet. The populations of active ingredient containing particles making up the composition of the invention may further be included in rapidly dissolving dosage forms such as an effervescent dosage form or a fast-melt dosage form.

The composition according to the invention comprises at least two populations of active ingredient containing particles which have different in vitro dissolution profiles.

Preferably, in operation, the composition of the invention and the solid oral dosage forms containing the composition release the active ingredient such that substantially all of the active ingredient contained in the first component is released prior to release of the active ingredient from the second component. When the first component comprises an IR component, for example, it is preferable that release of the active ingredient from the second component is delayed until substantially all the active ingredient in the IR component has been released. Release of the active ingredient from the second component may be delayed as detailed above by the use of a modified-release coating and/or a modified release matrix material.

The term “particulate” as used herein refers to a state, of matter which is characterized by the presence of discrete particles, pellets, beads or granules irrespective of their size, shape or morphology. The term “multiparticulate” as used herein means a plurality of discrete, or aggregated, particles, pellets, beads, granules or mixture thereof irrespective of their size, shape or morphology.

The term “modified release” as used herein in relation to the composition according to the invention or a coating or coating material or used in any other context means release which is not immediate release and is taken to encompass controlled release, sustained release and delayed release.

The term “time delay” as used herein refers to the duration of time between administration of the composition and the release of the active ingredient from a particular component of the composition.

The term “lag time” as used herein refers to the time between delivery of active ingredient from one component and the subsequent delivery of active ingredient from another component.

Although embodiments of the present invention are not limited to any particular mechanism, many physiological factors influence the release of a drug from a controlled-release dosage form and, thus, influence the uptake of the drug into the systemic circulation. Dosage forms should, therefore, be designed so that such variable factors do not compromise the efficacy and safety of the product. In some applications, such controlled-release dosage forms should release the active pharmaceutical ingredient at a controlled rate such that the amount of active pharmaceutical ingredient which is available in the body to treat the condition is maintained at a relatively constant level over an extended period of time. That is, the active pharmaceutical ingredient is released at a reproducible, predictable rate which is substantially independent of physiological factors which can vary considerably among different individuals and even over time for a particular individual.

A rational approach to obtain detailed information about how well topiramate is absorbed from the key residence areas of the gastrointestinal tract was conducted using the Enterion® site-specific delivery capsule, which is the subject of U.S. Pat. No. 6,632,216 which is incorporated herein by reference. The Enterion® site-specific delivery capsule is an easy to use non-invasive methodology for assessing regional drug absorption from the gastrointestinal tract. The capsule is 35mm in length and 10-12 mm in diameter and is capable of delivering solutions, suspensions or powders to specific sites. The location of the capsule in the gastrointestinal tract is determined using gamma scintigraphy, thereby resulting in significantly lower radiation doses to the volunteers. This results in more accurate assessment of anatomical location. The capsules contain a drug chamber with a wide port, and therefore loading of all types of drug formulations into the capsule is relatively straightforward. Capsule activation is confirmed by means of a signal, which is emitted from the capsule when activation occurs and is relayed back to the activation unit.

A pharmacoscintigraphy study of topiramate was conducted at Pharmaceutical Profiles, Nottingham, UK utilizing their Enterion® site-specific delivery capsule technology. This study was designed to obtain detailed information about how well topiramate is absorbed from the key residence areas of the gastrointestinal tract. The examples below are a result of a biostudy that was a randomized, 4-treatment, 4-period study, conducted in 12 healthy volunteers. The test treatments administered using an Enterion® capsule and were as follows: Trt B represents 200 mg topiramate API delivered to the proximal small bowel (PSB), Trt C represents 200 mg topiramate API delivered to the distal small bowel (DSB), Trt D represents 200 mg topiramate API delivered to the colon. The reference treatment administered was Trt A which represents 200 mg topiramate as the commercial immediate release reference formulation, Topamax™. Topiramate plasma concentrations were measured in this study.

Analysis of plasma samples for topiramate was carried out in accordance with Bioanalytical Services Test Method number TM154. The method involves solid phase extraction of topiramate using Oasis HLB extraction cartridges. Topiramate is analysed using LC-MS/MS.

The standard curves for the analytical runs in the study covered the concentration range of 10-1000 ng/mL with a limit of quantitation of 10 ng/mL. All samples were diluted 1:10 prior to analysis.

The following parameters were derived from the plasma concentration data: individual and mean blood drug concentration levels, individual and mean peak levels, Cmax; individual and mean AUClast and AUCinf; individual and mean time to peak concentration, tmax, relative bioavailability of test compared to alternative formulation, Frel (%).

TABLE 1
Mean Pharmacokinetic Parameter Table (Mean ± SD − CV %)

| | PK Parameters | | | |
|---|---|---|---|---|
| | | Trt B | Trt C | |
| | Trt A | 200 mg Topiramate | 200 mg Topiramate | Trt D |
| | 200 mg Topamax ™ | Proximal Small Bowel | Distal Small Bowel | 200 mg Topiramate |
| | IR | (PSB) | (DSB) | Colon |
| | n11 | n12 | n11 | n11 |
| Relative Bioavailability (%) | — | 120.88 ± 20.20* | 120.99 ± 25.31$ | 111.85 ± 19.41\hat{ } |
| Based on AUCinf CV % | | 16.7 | 20.9 | 17.4 |
| Relative Bioavailability (%) | — | 113.06 ± 19.46# | 125.69 ± 27.18˜ | 107.14 ± 22.73˜ |
| Based on AUClast CV % | | 17.2 | 21.6 | 21.2 |
| AUCinf (ng/mL · h) | 156940.46 ± 28400.34$ | 192140.44 ± 33798.87˜ | 193203.21 ± 57012.26 | 181462.53 ± 46104.28* |
| CV % | 18.1 | 17.6 | 29.5 | 25.4 |
| AUClast (ng/mL · h) | 126628.94 ± 19008.19 | 150405.64 ± 37858.89 | 160439.04 ± 38522.08 | 140008.32 ± 40685.00 |
| CV % | 15.0 | 25.2 | 24.0 | 29.1 |
| Cmax (ng/mL) | 4546.59 ± 781.39 | 5255.66 ± 1870.32 | 4972.90 ± 1200.60 | 3878.07 ± 1267.71 |
| CV % | 17.2 | 35.6 | 24.1 | 32.7 |
| tmax (h) | 2.14 ± 1.75 | 2.08 ± 1.08 | 3.64 ± 4.20 | 11.85 ± 7.44 |
| CV % | 81.7 | 51.8 | 115.4 | 62.8 |
| t½ (h) | 30.36 ± 8.13$ | 32.70 ± 9.70˜ | 24.52 ± 6.18 | 24.92 ± 5.20* |
| CV % | 26.8 | 29.7 | 74.3 | 20.9 |
| Lambda (h⁻¹) | 0.03 ± 0.01$ | 0.02 ± 0.01˜ | 0.03 ± 0.01 | 0.03 ± 0.01* |
| CV % | 30.2 | 29.3 | 27.2 | 24.2 |

*n = 7
$n = 9
\hat{ }n = 6
˜n = 10
#n = 11

Results

An increase in systemic availability as determined by AUCinf were observed for the test treatments (Trt A −192140.4±33798.9ng/mL.h) (Trt B −193203.2±57012.3ng/mL.h) (Trt C −181462.5±46104.3 ng/mL.h) as compared to the reference product, Topamax™ (Trt A −156940.5±28400.3 ng/mL.h).

There was an increase in the maximum concentration value (Cmax) observed for the two test treatments (Trt B −5255.7±1870.3 ng/mL) (Trt C −4972.9±1200.6 ng/mL) as compared to the reference treatment, (Trt A −4546.6±781.4 ng/mL).

A decrease in the Cmax was observed for the test treatment D (Colon) (3878.1±1267.7 ng/mL) as compared to the reference, treatment Trt A.

The time to reach maximum concentration (tmax) increased in order across the release sites, Trt B (PSB) (2.1±1.1 h), Trt C (DSB) (3.6±4.2 h), Trt D (Colon) (11.8±7.4 h), compared to the reference Topamax™ IR (2.1±1.7 h).

The activation of the Enterion® capsule and consequent release of the topiramate API in the proximal and distal small bowel regions resulted in the comparably enhanced systemic availability of topiramate compared to release in the colon or oral administration of immediate release tablets.

The results of this study demonstrated that there was enhanced bioavailability of topiramate (approximately 20%) if administered either in the small or large intestine as compared to oral administration of immediate release tablets. Thus suggesting the achievability of a once daily dosage form of topiramate.

The mean plasma concentration data from the orally administered dose of topiramate was then fitted to a 1 or 2 compartmental model to determine the pharmacokinetic parameters for topiramate following oral administration. The two compartmental model was selected as the most appropriate model and was used for simulation of various types of controlled release dosage formulations.

First order release target profiles were generated using a Ka^{rel} in the range of 0.12 to 0.3 l/hr. It was assumed that the release rate from the formulation (Ka^{rel}) is the controlling rate constant for absorption. Following simulation using a Ka^{rel} in the range above, the predicted steady state PK parameters show that the greatest degree of fluctuation occurs following administration of topiramate (56.82%) as compared to the different release rate formulations which had % fluctuations ranging from 19.4-34.8%.

Target profiles were also generated assuming zero order delivery over 8, 14 or 20 hours. This corresponds to a release of 12.5%, 7.14% or 5% per hour. The % fluctuation following simulation of the zero order release formulations were reduced (11.2-40.6%) compared to topiramate (56.8%).

Target profiles were also generated using pulsatile delivery in which a second immediate release pulse of equal amount absorbed occurred from 6 to 20 hours after the initial pulse. The predicted steady state PK parameters show that the greatest degree of fluctuation occurs following administration of topiramate (52.11%) as compared to the bipulsatile simulations, which had % fluctuations ranging from 26.25-44.35%.

Target profiles were also generated using pulsatile delivery in which the second immediate release pulse of 20% higher absorption occurred from 6 to 20 hours after the initial pulse. The predicted steady state PK parameters show that the greatest degree of fluctuation occurs following administration of topiramate (52.11%) as compared to the bipulsatile simulations, which had % fluctuations ranging from 28.69-43.99 Therefore, a formulation with first order release in the range of about 0.12 to about 0.3 l/hr, zero order release of about 5 to about 12.5% per hour, or a bipulsatile formulation with the second pulse occurring from about 6 to about 20 hours after the first pulse of topiramate would result in a smoother steady state profile than the currently available product and consequently would maintain therapeutic levels for a longer period over the dosing interval. This would allow for once a day dosing to improve patient compliance and may result in a reduced adverse event profile due to minimizing percent fluctuation.

We claim:

1. A controlled-release pharmaceutical composition comprising topiramate which is capable of delivering therapeutic amounts of topiramate to a specific region of the gastrointestinal tract of an animal.

2. The composition of claim 1 wherein the animal is a human.

3. The composition of claim 1 wherein the specific region of the gastrointestinal tract is the proximal small bowel, distal small bowel or colon.

4. A controlled-release pharmaceutical composition comprising: (A) an immediate-release component comprising from about 5 mg to about 250 mg of topiramate which is released in the body within about 1 hour after administration; and (B) a delayed-release component comprising from about 5 mg to about 250 mg of topiramate which is released in the body over a period of time of about 6 hours to 24 hours after administration.

5. An oral dosage form comprising the composition of claim 4.

6. The oral dosage form of claim 5 wherein the dosage is designed to be administered as a once-daily formulation.

7. The composition of claim 4 wherein the delayed-release component is achieved by the use of pharmaceutically acceptable erodable formulation.

8. The composition of claim 4 wherein the delayed-release component is achieved by the use of a pharmaceutically acceptable diffusion controlled formulation.

9. The composition of claim 4 wherein the delayed-release component is achieved by the use of a pharmaceutically acceptable osmotically controlled formulation.

10. An oral controlled-release pharmaceutical composition comprising topiramate which is designed to deliver therapeutic amounts of topiramate to the colonic region of a gastrointestinal tract at lower doses than other regions of the gastrointestinal tract with substantially equal bioavailability.

11. The composition of claim 8 which comprises the following components, each of which includes topiramate: (A) an immediate-release component comprising from about 5 mg to about 250 mg of topiramate which is released as a pulse within about 1 hour after ingestion; and (B) a delayed-release component comprising from about 5 mg to about 250 mg of topiramate which is released as a pulse at about 6 hours, about 9 hours, about 12 hours or about 18 hours after the release of the immediate-release component.

12. The composition of claim 9 which further comprises at least one additional delayed-release component comprising from about 5 mg to about 250 mg of topiramate which is released as a pulse at about 6 hours, about 9 hours, about 12 hours or about 18 hours after the release of the first delayed-release component.

13. A solid oral dosage form comprising the composition of claim 8.

14. The solid oral dosage form of claim 10 wherein the dosage is administered as a once-daily formulation.

15. A method of treating an animal for partial seizures with or without secondarily generalized seizures, primary generalized tonic-clonic seizures, seizures associated with Lennox Gastaut Syndrome or migraines comprising the step of administering the composition of claim 1.

16. The composition of claim 1 further comprising a sympathomimetic agent.

17. The composition of claim 16 wherein the sympathomimetic agent is phentermine.

18. The composition of claim 4 further comprising a sympathomimetic agent.

19. An oral dosage form comprising the composition of claim 18.

20. The oral dosage form of claim 19 wherein the dosage is designed to be administered as a once-daily formulation.
